(19) United States
(12) Patent Application Publication
(10) Pub. No.: US 2024/0368201 A1
(43) Pub. Date: Nov. 7, 2024

(54) SILANE HYDROLYZATE AND PROCESSES FOR THE PREPARATION AND USE THEREOF

(71) Applicants: Dow Silicones Corporation, Midland, MI (US); Dow Global Technologies LLC, Midland, MI (US)

(72) Inventors: Lorry Deklippel, Pieton (BE); Cindy Delvalle, Seneffe (BE); Eric Joffre, Midland, MI (US); Axel Kretschmer, Braine l'Alleud (BE); Jeremy Marchand, Midland, MI (US); Donald Eldred, Midland, MI (US); Cheryl Roggenbuck, Midland, MI (US); Xiaoyun Chen, Midland, MI (US); Joe Nowell, Midland, MI (US); Na Li, Midland, MI (US); Giuseppina Lavinaro, Seneffe (BE)

(21) Appl. No.: 18/683,346

(22) PCT Filed: Oct. 10, 2022
(86) PCT No.: PCT/US22/77830

§ 371 Date: Feb. 13, 2024

Related U.S. Application Data
(60) Provisional application No. 63/282,196, filed on Nov. 23, 2021.

(51) Int. Cl. C07F 7/18; A61K 8/58; A61Q 5/06

(52) U.S. Cl. CPC C07F 7/188; A61K 8/585; A61Q 5/06; A61K 2800/805

(57) ABSTRACT

A carbamate anion/protonated amine-functional silane hydrolyzate is prepared by a process including combining alkoxysilanes, base catalyst and water to form a silane hydrolyzate, and thereafter combining the silane hydrolyzate with carbon dioxide. The resulting carbamate anion/protonated amine-functional silane hydrolyzate is suitable for use in hair care compositions.

### CROSS REFERENCE TO RELATED APPLICATIONS

This application claims the benefit of U.S. Provisional Application Ser. No. 63/282,196 filed on 23 Nov. 2021 under 35 U.S.C. 119(e). U.S. Provisional Application Ser. No. 63/282,196 is hereby incorporated by reference.

### FIELD

A silane hydrolyzate and processes for its preparation and use are provided. More particularly, the silane hydrolyzate has both carbamate anion and protonated amine functionalities. The silane hydrolyzate can be prepared by combining alkoxysilanes, catalyst, and water, and thereafter neutralizing with carbon dioxide. The resulting silane hydrolyzate is suitable for use in hair care compositions.

### INTRODUCTION

U.S. Pat. No. 9,962,327 discloses a cosmetic or dermatological composition of sol/gel type for making up and/or caring for keratin materials. This composition is obtained by sol/gel reaction by mixing at least one alkoxysilane and at least one specific amount of water, determined according to the number of moles of alkoxy groups in the mixture. The alkoxysilane may have an amino group.

However, excessive amounts of amino groups can impart undesirable odor to cosmetic or dermatological compositions, such as those used for hair care applications. In addition, amino groups may act as skin irritants and/or sensitizers. Therefore, there is a need in the silicones industry for silane hydrolyzates with reduced amino content suitable for use in cosmetic or other personal care compositions that may contact skin, such as hair care compositions that may also contact skin, e.g., the scalp.

### SUMMARY

A carbamate anion/protonated amine-functional silane hydrolyzate may be prepared by a process comprising:

(1) combining, at a temperature up to 85° C., starting materials (A), (B), (C), and (D), wherein
starting material (A) is an amino-functional alkoxysilane compound;
starting material (B) is an organoalkoxysilane compound;
where (A) the amino-functional alkoxysilane compound and (B) the organoalkoxysilane compound are used in a weight ratio (B)/(A) of 1 to 3.5;
starting material (C) is a base catalyst; and
starting material (D) is water, where (D) the water is used in an amount corresponding to a number of moles determined according to the following equation
X=(n_{alkoxysilane},xnb_{alkoxygroups})/n_{H2O}, where
n_{H2O}=number of moles of water,
n_{Alkoxysilanes}=number of moles of alkoxysilane compounds (A) and (B);
nb_{alkoxygroups}=weighted mean of the number of alkoxysilane groups per alkoxysilane compound, and
X≥2.5;
thereby forming a silane hydrolyzate comprising unreacted (A) amino-functional alkoxysilane compound and/or unreacted (B) organoalkoxysilane compound, an organosiloxane oligomer, and an alcohol; and
   (2) combining, at a temperature up to 85° C., the silane hydrolyzate and (E) carbon dioxide, thereby neutralizing (C) the base catalyst and forming a reaction product comprising the carbamate anion/protonated amine-functional silane hydrolyzate.

### DETAILED DESCRIPTION

In step (1) of the process introduced above, starting materials (A), (B), (C), and (D) may be combined by any convenient means, such as mixing optionally with heating. Combining may be performed in any convenient equipment, such as a batch reactor equipped with an agitator and heating and cooling means, such as a jacket. The starting materials may be combined in any order.

The starting materials may be combined at RT, alternatively elevated temperature. The temperature depends on various factors including the boiling points of the alkoxysilane compounds selected as starting materials (A) and (B) and the desired reaction time. However, the starting materials may be combined at a temperature of at least 20° C., alternatively at least 25° C., alternatively at least 50° C., while at the same time the temperature may be up to 85° C., alternatively up to 80° C., and alternatively up to 75° C. Alternatively, the temperature in step (1) may be 20° C. to 85° C., and alternatively 50° C. to 85° C.

In step (1), the starting materials may be combined under an inert atmosphere, e.g., nitrogen. Without wishing to be bound by theory, it is thought that addition of a solvent, such as a monohydric alcohol (e.g., methanol or ethanol) and/or agitation may be employed to minimize or eliminate formation of a gel side product in step (1).

Step (1) may be performed for a time sufficient for all, or substantially all, of (D) the water to be consumed. The time depends on various factors including the reactivities of the alkoxysilane compounds selected as starting materials (A) and (B), the amount of (C) the base catalyst, and the temperature, however, the time for step (1) may be at least 1 hour, alternatively at least 2 hours, alternatively at least 4 hours, while concurrently, the reaction time may be up to 48 hours, alternatively up to 36 hours, alternatively up to 24 hours. Alternatively, the reaction time in step (1) may be 1 hour to 48 hours, alternatively 1 hour to 36 hours, alternatively 1 hour to 24 hours, alternatively 2 hours to 22 hours, alternatively 4 hours to 24 hours, and alternatively 4 hours to 22 hours.

The resulting silane hydrolyzate prepared in step (1) comprises a mixture of unreacted alkoxysilane compound (A) and/or (B), an organosiloxane oligomer, and an alcohol, which is a side product of the reaction of (D) the water and the alkoxy groups of starting materials (A) and (B).

In step (2) of the process, the silane hydrolyzate and (E) the carbon dioxide may be combined at RT, alternatively elevated temperature. Alternatively, (E) the carbon dioxide may be below RT, for example, in step (2) cardice may be added to the silane hydrolyzate prepared in step (1). The temperature depends on various factors including the form of (E) the carbon dioxide and the desired reaction time. However, the step (2) may be performed at a temperature of at least 20° C., alternatively at least 25° C., alternatively at least 50° C., and alternatively at least 70° C., while at the same time the temperature may be up to 85° C., alternatively up to 80° C., and alternatively up to 75° C. Alternatively, the temperature in step (2) may be 20° C. to 85° C., and alternatively 50° C. to 85° C., and alternatively 70° C. to 85° C.

During or after step (2), pressure may be reduced and/or heat may be increased to at least 70 C. (if step (2) was begun at a lower temperature). Without wishing to be bound by theory, it is thought that this will remove dissolved carbon dioxide and minimize or prevent over pressurization after step (2).

The process described herein may optionally further comprise one or more additional steps. For example (C) the base catalyst may optionally be dissolved in water, before combining starting materials (A), (B), (C), and (D) in step (1). The base catalyst may be dissolved in the water used as starting material (D) or the base may be supplied as an aqueous solution with known molarity.

The process described above may further comprise a solvent exchange step after step (2). For example, if a solvent other than an alcohol (e.g., methanol or ethanol) is desired, all or a portion of the alcohol (produced as a side product in step (1) and/or otherwise added during the process) may be removed and replaced with a different solvent, such as a polyorganosiloxane or an organic emollient. Exemplary polyorganosiloxanes include linear polydimethylsiloxanes such as those with tradename DOWSIL™ OS Fluids and cyclic polydimethylsiloxanes such as octamethylcyclotetrasiloxane (D4), decamethylcyclopentasiloxane (D5), and/or dodecamethylcyclohexasiloxane (D6). The DOWSIL™ OS Fluids and D4, D5, and D6 are known in the art and are commercially available, e.g., from DSC.

Alternatively, the carbamate anion/protonated amine-functional silane hydrolyzate produced in step (2) of the process described above may be concentrated by removing all or a portion of the alcohol. Alcohol removal may be performed by any convenient means such as stripping and/or distillation.

Alternatively, the process may further comprise treating the carbamate anion/protonated amine-functional silane hydrolyzate produced as described above with an adsorbent, such as activated carbon, which may remove impurities, such as undesired color. Alternatively, the process may further comprise filtering the carbamate anion/protonated amine-functional silane hydrolyzate produced as described above to remove side products such as a gel and/or to remove activated carbon, when activated carbon is used.

The starting materials used in the process are described in detail, below.

### (A) Amino-Functional Alkoxysilane Compound

In step (1) of the process described above, starting material (A) may be an amino-functional alkoxysilane compound of formula (A1): R¹R³₍₃₋ₐ₎Si(OR²)ₐ, where each R¹ is an independently selected amino-alkyl group, each R² is an independently selected alkyl group, each R³ is an independently selected monovalent hydrocarbon group, and subscript a is an integer from 1 to 3. Alternatively, subscript a may be 2 or 3. Alternatively, subscript a may be 3. Suitable amino-alkyl groups for R¹ may have formula —(C_{c}H_{2c})NH₂, where subscript c is 1 to 20, alternatively 2 to 12, alternatively 3 to 8, and alternatively c=3. Alternatively, R¹ may be amino-propyl.

R² is an alkyl group, which may have formula —C_{d}H_{(2d+1)}, where subscript d is 1 to 10, alternatively 1 to 6, and alternatively 1 to 4. The alkyl groups for R² are exemplified by methyl, ethyl, propyl (including n-propyl and/or iso-propyl), butyl (including n-butyl, t-butyl, sec-butyl, and/or isobutyl), pentyl (including n-pentyl and branched alkyl groups of 5 carbon atoms), and hexyl (including n-hexyl and branched alkyl groups of 6 carbon atoms). Alternatively, each R² may be independently selected from the group consisting of methyl, ethyl, propyl, and butyl. Alternatively, each R² may be independently selected from the group consisting of methyl and ethyl. Alternatively, each R² in (A) the amino-functional alkoxysilane compound may be ethyl.

The monovalent hydrocarbon group for R³ may be an alkyl group, an alkenyl group, or an aryl group. The alkyl group may have 1 to 20 carbon atoms, alternatively 1 to 12, carbon atoms, alternatively 1 to 10 carbon atoms, and alternatively 1 to 6 carbon atoms. The alkyl groups for R³ are exemplified by methyl, ethyl, propyl (including n-propyl and/or iso-propyl), butyl (including n-butyl, t-butyl, sec-butyl, and/or isobutyl), pentyl (including n-pentyl and branched alkyl groups of 5 carbon atoms), and hexyl (including n-hexyl and branched alkyl groups of 6 carbon atoms). Alternatively, the alkyl group may be independently selected from the group consisting of methyl, ethyl, propyl, and isobutyl. Alternatively, the alkyl group for R³ may be independently selected from the group consisting of methyl, ethyl, and isobutyl. Alternatively, the alkyl group for R³ may be independently selected from the group consisting of methyl and ethyl. Alternatively, each R³ in (A) the amino-functional alkoxysilane compound may be methyl. Suitable alkenyl groups for R³ include vinyl, allyl, and hexenyl. Suitable aryl groups for R³ include phenyl, tolyl, xylyl, and benzyl; alternatively phenyl. Suitable halogenated hydrocarbon groups for R³ include any of the alkyl, alkenyl, and aryl groups described above, wherein one or more hydrogen atoms has been replaced with a halogen atom, such as Cl or F. For example R³ may be a haloalkyl group such as chloromethyl or trifluoromethyl. Alternatively, each R³ may be an alkyl group, as described above.

Amino-functional alkoxysilane compounds suitable for use herein are known in the art and commercially available. For example, 3-aminopropyltriethoxysilane (APTES), 3-aminopropylmethyldiethoxysilane (APMDES), 3-aminopropyltrimethoxysilane (APTMS), 3-aminopropylmethyldimethoxysilane (APMDMS), or a combination of two or more thereof. Amino-functional alkoxysilane compounds are commercially available from various sources, for example, APTES is available from DSC under the name XIAMETER™ OFS-6011 Silane. APMDES is available, for example, from Evonik of Germany, under the name DYNASYLAN™ 1505.

Alternatively, (A) the amino-functional alkoxysilane compound may be an amino-functional trialkoxysilane of formula (A2): R¹Si(OR²)₃, where R¹ and R² are as described above. Alternatively, the amino-functional trialkoxysilane may be selected from the group consisting of APTES, APTMS, and a combination thereof. Alternatively, the amino-functional trialkoxysilane may be APTES.

### (B) Organoalkoxysilane Compound

In the method described above, starting material (B) is an organoalkoxysilane compound of formula (B1): R³_{(4-b)}Si(OR²)_{b}, where each R³ is independently selected from the group consisting of a monovalent hydrocarbon group and a monovalent halogenated hydrocarbon group as described above, each R² is an independently selected alkyl group, and subscript b is an integer from 1 to 3. Alternatively, subscript b may be 2 or 3, and alternatively subscript b=3.

The monovalent hydrocarbon group for R³ in starting material (B) is as described above for starting material (A). Alternatively, the monovalent hydrocarbon group for R³ in starting material (B) may be an alkyl group, alternatively methyl, ethyl, propyl, or butyl. Alternatively, R³ in starting material (B) may be selected from the group consisting of methyl, ethyl, and isobutyl. Alternatively, R³ in starting material (B) may be selected from the group consisting of methyl and isobutyl.

In the organoalkoxysilane compound of formula R³_{(4-b)}Si(OR²)_{b}, each R² is an independently selected alkyl group. The alkyl groups for R² are exemplified by methyl, ethyl, propyl (including n-propyl and/or iso-propyl), butyl (including n-butyl, t-butyl, sec-butyl, and/or isobutyl), pentyl (including n-pentyl and branched alkyl groups of 5 carbon atoms), and hexyl (including n-hexyl and branched alkyl groups of 6 carbon atoms). Alternatively, each R² may be independently selected from the group consisting of methyl, ethyl, propyl, butyl. Alternatively, each R² may be independently selected from the group consisting of methyl and ethyl. Alternatively, each R² in (B) the organoalkoxysilane compound may be methyl.

Examples of suitable (B) organoalkoxysilane compounds for use herein include methyltrimethoxysilane (MTMS), methyltriethoxysilane (MTES), dimethyldiethoxysilane (DMDES), ethyltrimethoxysilane (ETMS), ethyltriethoxysilane (ETES), diethyldiethoxysilane (DEDES), dipropyldiethoxysilane (DPDES), propyltrimethoxysilane (PTMS), propyltriethoxysilane (PTES), butyltrimethoxysilane, e.g., isobutyltrimethoxysilane (IBTMS), butyltriethoxysilane, e.g., isobutyltriethoxysilane (IBTES), phenyltriethoxysilane, phenylmethyldiethoxysilane, diphenyldiethoxysilane, benzyltriethoxysilane, benzylmethyldiethoxysilane, dibenzyldiethoxysilane, and combinations of two or more thereof.

Organoalkoxysilane compounds are known in the art and are commercially available from various sources such as DSC, Evonik, Gelest, Inc. of Morrisville, Pennsylvania USA. For example, methyltrimethoxysilane, propyltrimethoxysilane, isobutyltrimethoxysilane, and isobutyltriethoxysilane are available under the tradename XIAMETER™ OFS from DSC. Methyltriethoxysilane is available under the tradename DYNASYLAN™ MTES and methyltrimethoxysilane is available under the tradename DYNASYLAN™ MTMS, both from Evonik. Isobutyltrimethoxysilane is available from Gelest.

Alternatively, (B) the organoalkoxysilane compound may be an organotrialkoxysilane. The organotrialkoxysilane may have formula (B2): R³Si(OR²)₃, where R³ and R² are as described above. For example, the organotrialkoxysilane may be selected from the group consisting of MTMS, MTES, ETMS, ETES, PTMS, PTES, IBTMS, IBTES, and a combination of two or more thereof.

In step (1) of the process described herein, (A) the amino-functional alkoxysilane compound and (B) the organoalkoxysilane compound are used in a weight ratio (B)/(A) of at least 1, alternatively at least 1.15, and alternatively at least 1.25, while at the same time the weight ratio (B)/(A) may be up to 3.5, alternatively up to 3, alternatively up to 2.5, and alternatively up to 2. Alternatively, the weight ratio (B)/(A) may be 1 to 3.5, alternatively 1.25 to 2.

### (C) Base Catalyst

In the process described herein, starting material (C) is a base catalyst. Examples include metal hydroxides such as sodium hydroxide (NaOH) and potassium hydroxide (KOH), metal acetates such as sodium acetate and potassium acetate, alkylammonium hydroxides such as trimethylammonium hydroxide and cetyltrimethylammonium hydroxide. Base catalysts are known in the art and are commercially available from various sources, e.g., Sigma-Aldrich, Inc. of St. Louis, Missouri, USA. The amount of (C) the base catalyst may be at least 0.01%, alternatively at least 0.02%, and alternatively at least 0.03%, while at the same time the amount may be up to 2%, alternatively up to 1%, alternatively up to 0.1%, and alternatively up to 0.075%, based on combined weights of all starting materials in step (1). The base catalyst may be supplied in an aqueous solution.

### (D) Water

In the process described herein, starting material (D) is water. The water is not generally limited, and may be utilized neat (i.e., absent any carrier vehicles/solvents), and/or pure (i.e., free from or substantially free from minerals and/or other impurities). For example, the water may be processed or unprocessed when used in step (1). Examples of processes that may be used for purifying the water include distilling, filtering, deionizing, and combinations of two or more thereof, such that the water may be deionized, distilled, and/or filtered. Alternatively, the water may be unprocessed (e.g. may be tap water, i.e., provided by a municipal water system or well water, used without further purification).

The amount of water corresponds to a number of moles determined according to the following equation

$$X = ( {n}_{alkoxysilane} , {xnb}_{alkoxygroups} ) / {n}_{H 2 O} ,$$

n_{H2O}=number of moles of water,
n_{Alkoxysilanes}=number of moles of alkoxysilane compounds (A) and (B);
nb_{alkoxygroups}=weighted mean of the number of alkoxysilane groups per alkoxysilane compound, where X≥2.5. Alternatively, X may be at least 2.5, alternatively at least 3, and alternatively at least 3.5, while at the same time X may be up to 500, alternatively up to 50, alternatively up to 4.11. Alternatively, X may be 2.5 to 500, alternatively 2.5 to 2.5 to 50, alternatively 2.5 to 4.11, alternatively 3 to 4.11, and alternatively 3.5 to 4.11. X can be calculated according to the method described in U.S. Pat. No. 9,962,327, but where X has the values recited hereinabove for the process of this invention.

### (E) Carbon Dioxide

In the method introduced above, starting material (E) is carbon dioxide. Carbon dioxide may be in gaseous or solid (cardice) form. Carbon dioxide is known in the art and commercially available from various sources, such as Air Products of Allentown, Pennsylvania, USA. The carbon dioxide is used in a molar excess with respect to (C) the base catalyst and the amino-functional groups from (A) the amino-functional alkoxysilane compound. However, the amount of carbon dioxide used may be at least 3.5%, alternatively at least 5%, while at the same time the amount may be up to 7.5%, alternatively up to 5%, based on weight of the silane hydrolyzate prepared in step (1). Alternatively, the amount of carbon dioxide may be 3.5% to 7.5% on the same basis.

### Carbamate Anion/Protonated Amine-Functional Silane Hydrolyzate

The carbamate anion/protonated amine (CAPA)-functional silane hydrolyzate, which can be prepared by the process described above, comprises a combination of species. The (CAPA)-functional silane hydrolyzate comprises a species of unit formula (I-1):

(R³Z₂SiO_{1/2})ₑ(R⁵Z₂SiO_{1/2})_{f}(R³ZSiO_{2/2})_{g}(R⁵ZSiO_{2/2})ₕ(R³SiO_{3/2})ᵢ(R⁵SiO_{3/2})ⱼ(R³R⁵ZSiO_{1/2})ₖ(R³R⁵Si O_{2/2})ₘ(R³₂ZSiO_{1/2})ₙ(R³₂SiO_{2/2})ₒ(R³₂SiO_{1/2})ₚ(R³₂R⁵SiO_{1/2})_{q}, where
each R³ is independently selected from the group consisting of a monovalent hydrocarbon group and a monovalent halogenated hydrocarbon group (as described and exemplified above);
each Z is an alkoxy group of formula OR², where each R² is an independently selected alkyl group (as described and exemplified above);
each R⁵ is independently selected from the group consisting of amino-alkyl, carbamate-alkyl, and protonated amine alkyl, with the proviso that at least one instance of R⁵ being a carbamate-alkyl and at least one instance of R⁵ being a protonated amine alkyl are present in the CAPA-functional silane hydrolyzate;
subscripts e, f, g, h, i, j, k, m, n, o, p, and q represent average numbers of each unit per molecule and have values such that e≥0, f≥0, g≥0, h≥0, i≥0, j≥0, k≥0, m≥0, n≥0, o≥0, p≥0, q≥0, a quantity (e+f+g+h+i+j+k+m+n+o+p+q)≥2, a quantity (e+g+i)>0, and a quantity (f+h+j+k+m)>0. The quantity (e+f+g+h+i+j+k+m+n+o+p+q) may have a value sufficient to give the species a molecular weight up to 2,000 g/mol. The CAPA-functional silane hydrolyzate is free of, or substantially free, of siloxane units of formula (SiO_{4/2}). The CAPA-functional silane hydrolyzate comprises a combination of different species.

Alternatively, when (A) the amino-functional alkoxysilane compound is an amino-functional trialkoxysilane, and when (B) the organoalkoxysilane compound is an organotrialkoxysilane, the CAPA-functional silane hydrolyzate may comprise species each having unit formula (I-2):

(R³Z₂SiO_{1/2})ₑ(R⁵Z₂SiO_{1/2})_{f}(R³ZSiO_{2/2})_{g}(R⁵ZSiO_{2/2})ₕ(R³SiO_{3/2})ᵢ(R⁵SiO_{3/2})ⱼ, where
each R³ is independently selected from the group consisting of a monovalent hydrocarbon group and a monovalent halogenated hydrocarbon group (as described and exemplified above);
each Z is an alkoxy group of formula OR², where each R² is an independently selected alkyl group (as described and exemplified above);
each R⁵ is independently selected from the group consisting of amino-alkyl, carbamate-alkyl, and protonated amine alkyl, with the proviso that at least one instance of R⁵ being a carbamate-alkyl and at least one instance of R⁵ being a protonated amine alkyl are present in the CAPA-functional silane hydrolyzate;
subscripts e, f, g, h, i, and j represent average numbers of each unit per molecule and have values such that e≥0, f≥0, g≥0, h≥0, i≥0, j≥0, a quantity (e+f+g+h+i+j)≥2, a quantity (e+g+i)>0, and a quantity (f+h+j)>0. The quantity (e+f+g+h+i+j) may have a value sufficient to give the species a molecular weight up to 2,000 g/mol. The CAPA-functional silane hydrolyzate is free of, or substantially free, of siloxane units of formula (SiO_{4/2}). The CAPA-functional silane hydrolyzate comprises a combination of different species including: i) a dimer where in the unit formula (I-2) above subscripts g=h=i=j=0 and the dimer has unit formula (I-3): (R³Z₂SiO_{1/2})ₑ(R⁵Z₂SiO_{1/2})_{f}, where subscript e is 0 or 1, subscript f is 1 or 2, and a quantity (e+f)=2; and ii) a linear trimer where in the unit formula (I-2) above subscripts i=j=0, and the linear trimer has unit formula (I-4):
(R³Z₂SiO_{1/2})ₑ(R⁵Z₂SiO_{1/2})_{f}(R³ZSiO_{2/2})_{g}(R⁵ZSiO_{2/2})ₕ, where subscript e is 0, 1, or 2; subscript f is 0, 1, or 2; a quantity (e+f)=2, a quantity (g+h)=1, and a quantity (f+h)>0. In addition to the dimer and trimer exemplified above, the CAPA-functional silane hydrolyzate may further comprise iii) one or more additional branched and linear organosiloxane species, wherein said species has a molecular weight up to 2,000 g/mol.

In the formulas for the species in the CAPA-functional silane hydrolyzate shown above, each R⁵ is independently selected from the group consisting of amino-alkyl, carbamate-alkyl, and protonated amine alkyl, with the proviso that at least one instance of R⁵ being a carbamate-alkyl and at least one instance of R⁵ being a protonated amine alkyl are present in the CAPA-functional silane hydrolyzate. The instance of the carbamate-alkyl and the instance of protonated amine alkyl do not have to occur in the same molecule; they can be in different species making up the CAPA-functional silane hydrolyzate. The carbamate-alkyl group for R⁵ may have formula —(C_{c}H_{2c})NH—C(O)—O⁻, where subscript c is as described above. Alternatively, the carbamate-alkyl group for R⁵ may be

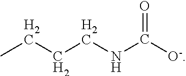

The protonated amine group for R⁵ may have formula —(C_{c}H_{2c})NH₃⁺, where subscript c is as described above. Alternatively, the protonated amine group for R⁵ may be

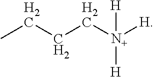

The amino-alkyl group for R⁵ may have formula —(C_{c}H_{2c})NH₂, where subscript c is as described above. Alternatively, the amino-alkyl group for R⁵ may be aminopropyl.

### Use of the CAPA-Functional Silane Hydrolyzate

The CAPA-functional silane hydrolyzate described herein may be used in various personal care applications, such as: i) the kit and process for dyeing keratinaceous material (particularly human hair) disclosed in U.S. Patent Application Publication 2021/0290512 in place of the organic silicon compound described therein; ii) the cosmetic compositions described in U.S. Patent Application Publication 2011/0104085; and/or iii) the cosmetic composition, the coating kit, and the coating process disclosed in U.S. Pat. No. 9,962,327 in place of the sol/gel composition described therein.

Alternatively, the CAPA-functional silane hydrolyzate described herein may be used in a hair care composition. The hair care composition may be, for example, a shampoo, a conditioner, a styling composition, a hair rinse, a volume spray, a styling aid (e.g., for curl retention and/or frizz control), a hair foam, a hair gel, a setting composition, a hairspray, a mousse, a hair oil, or an ends fluid.

One or more additional starting materials may be added to the CAPA-functional silane hydrolyzate described herein in preparation of a personal care composition, such as a hair care composition. The one or more additional starting materials may be, for example, additional water and/or a silicone oil, e.g., a cyclic polydiorganosiloxane such as decamethylcyclopentasiloxane (D5), which is available from DSC. Other additional starting materials are known in the art and are commercially available, e.g., the additional compounds disclosed, for example in U.S. Patent Application Publication 2021/0290521; U.S. Patent Application Publication 2011/0104085; and/or U.S. Pat. No. 9,962,327.

### Examples

The following examples are intended to illustrate the invention to one skilled in the art and should not be interpreted to limit the scope of the invention set forth in the claims. The starting materials used herein are summarized in Table 1.

TABLE 1
Starting Materials

| Starting Material | Description | Source |
|---|---|---|
| APTES | Aminopropyltriethoxysilane | |
| MTES | Methyltriethoxysilane | |
| NaOH in water | Sodium hydroxide dissolved in water | |
| Cardice | Solid carbon dioxide | |
| CO₂ gas | Carbon dioxide gas | |
| D5 | decamethylcyclopentasiloxane | DSC |

In this Synthesis Example 1, methyltriethoxysilane, aminopropyltriethoxysilane, and a solution of NaOH in water (0.1 mol/L) were combined in a 4 necked flask containing a magnetic stirrer. The flask was placed on a heating system comprising a hot plate with heat-on block. One neck was connected to a nitrogen supply, another neck was fitted with a condenser (chilled at 1° C.) placed vertically and connected to a bubbling system, thereby enabling the flask and condenser to be flushed with nitrogen gas, one neck was fitted with a temperature probe, and one neck was stoppered. The contents of the flask were stirred and heated at 70° C. for 24 hours.

Half of the contents of the flask were removed and stored in a high density polyethylene bottle with a nitrogen blanket. This is Comparative Sample A.

The contents of the flask were cooled to 30° C., and 12.5 g of cardice (CO₂ solid) were added thereto to neutralize the NaOH sol, and the contents of the flask were analyzed by ¹³C NMR spectroscopy. The analysis showed 37.1% carbamate and 0.7% carbamic acid content, as well as the presence of carbamic acid [3-(triethoxysilyl)propyl] and 1-Propanamine, 3-(triethoxysilyl)-, polymer with triethoxymethylsilane hydrolyzed with carbamate. This is Working Sample B.

In this Synthesis Example 2, 154.2 g of methyltriethoxysilane and 78.09 g of aminopropyltriethoxysilane were combined with 18.74 g of 0.25 M NaOH solution in water in a 4 neck flask. The flask was placed in a heating block on a hot plate. A thermometer was placed in one neck to monitor the reaction mixture temperature. The center neck was connected to a vertical water cooled condensation column that also allowed for N₂ to enter the headspace providing an inert blanket over the reaction mixture. The two other necks were stoppered. One neck would be for sampling during the neutralization process and the other would be for CO₂ neutralization. Once the set up was complete, a magnetic stir bar was added and head space was inerted. Mixing was set to 600 rpm and a temperature controller was set to 80° C. Heating took an hour to reach temperature. The reaction mixture appeared hazy and was stirred at 80° C. for 22 hours. After 22 hours the reaction mixture appeared clear and heating was turned off. Once the reaction mixture reached room temperature, one stopper was replaced with a rubber stopper. A needle was inserted into the reaction mixture through the rubber stopper. The needle was connected to a small cylinder of compressed CO₂. Before the CO₂ gaseous feed was started, an initial 10 g sample was taken. CO₂ flow was then started, and a flow meter was used to control the flow rate to 18 m/min. A 10 g sample was then taken every 15 minutes for two hours. After two hours the heat and CO₂ were turned off, and the material in the flask cooled to room temp. The material was put in a glass bottle. Initially the material appeared clear and colorless. After 4 days, there was a color change.

The samples were evaluated by Near IR, which showed a correlation between exposure to CO₂ and reduction in the concentration of the amine moiety and subsequent increase in the protonated amine concentration. As CO₂ bubbling time increased, NH₂ content decreased to a minimum, and NH₃+ content increased, to a maximum after 50-100 minutes. FTIR analysis did not show the presence of a carbonyl peak, which was consistent with the formation of a carbamate anion. Carbamate anion-functional propyltriethoxysilane is shown.

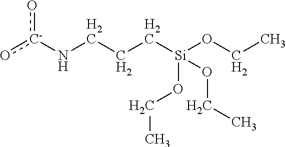

In this Synthesis Example 3, methyltriethoxysilane, aminopropyltriethoxysilane and NaOH solution in water (0.1 mol/L) were put into a 1,000 mL flask with a magnetic stir bar. The flask was placed on a heating system (hot plate with heat-on block). One neck of the flask was connected to nitrogen gas supply, and another neck was fitted with a condenser (chilled at 1° C.) placed vertically and connected to a bubbling system to be able to flush the flask and condenser with nitrogen gas. One neck was fitted with a temperature probe and one neck with a stopper. The contents of the flask were heated to 70° C. with stirring. When the temperature of 70° C. was reached, heating at 70° C. with stirring continued for 24 hours. Finally, the resulting silane hydrolyzate was divided into three parts.

One of the parts (194.67 g) was neutralized with 1.38 g of HCl solution (1.25 M HCl in EtOH). The quantity of HCl solution used was just to neutralize the NaOH solution. This is Comparative Sample C.

In this Example 4, Comparative Sample A and Working Sample B were tested for cure speed using an Anton Paar MCR300 Rheometer, according to the procedure described below. Working Sample B was found to cure faster than the Comparative Sample A under the conditions tested.

In this Example 5, Frizz Control was tested using Comparative Samples A and C and Working Sample B, according to the test methods described below. Working Sample B and Comparative Sample C had improved frizz control over the Comparative Sample A, and over an untreated hair control by both the Long Lasting Frizz Index and the Aspect Ratio measurements, which are as described below.

In this Example 6, Curl Retention percentage was tested, as described below, on samples containing D5 and one of Comparative Samples A and C, and Working Sample B. D5 alone and an untreated sample were also tested as controls. Working Sample B and Comparative Sample C were found to impart improved curl retention to hair, as compared to Comparative Sample A and also as compared to the controls. Results are shown below in Table 2

TABLE 2
Curl Retention Percentage

| | % Curl Retention | | | | | |
|---|---|---|---|---|---|---|
| Sample | 0 h (initial) | 1 h | 2 h | 4 h | 6 h | 8 h |
| Untreated control | 100 | 17 | 13 | 11 | 9 | 8 |
| D5 control | 100 | 25 | 20 | 18 | 15 | 12 |
| Working Sample B | 100 | 97 | 96 | 94 | 93 | 91 |
| Comparative Sample A | 100 | 96 | 92 | 90 | 83 | 81 |
| Comparative Sample C | 100 | 99 | 98 | 96 | 93 | 92 |

Without wishing to be bound by theory, it is thought that Working Sample B, which contained carbamate anion functionality, would have fewer aminopropyl moieties present than Comparative Sample C, which was neutralized with HCl. Therefore, Working Example B would also be expected to have less undesirable odor and less potential for skin irritation and/or sensitization when used in a hair care composition that may contact the scalp of a person using the hair care composition.

Additional samples were prepared according to the method of Synthesis Example 1, described above, but with varying the water ratio and carbamate level. The samples are summarized below in Table 3. In Table 3, “methyl” refers to methyltriethoxysilane, and “amino” refers to aminopropyltriethoxysilane. Additional examples for curl retention were performed as described below using materials summarized in Table 3, and the results are in Table 3a.

TABLE 3
Materials

| Name | Product |
|---|---|
| D5 (Comparative) | decamethylcyclopentasiloxane from DSC |
| Working Sample D | Water Ratio 3.5 |
| | (60:30 Methyl:Amino) |
| | Carbamate level 0.4% |
| Working Sample E | Water Ratio 3 |
| | (60:30 Methyl:Amino) |
| | Carbamate level 0.4% |
| Working Sample F | Water Ratio 4.11 |
| | (60:30 Methyl:Amino) |
| | Carbamate level 0.4% |
| Working Sample G | Water Ratio 3 |
| | (60:30 Methyl:Amino) |
| | Carbamate level 0.2% |
| Working Sample H | Water Ratio 3.5 |
| | (60:30 Methyl:Amino) |
| | Carbamate level 0.2% |

TABLE 3a

| | % curl retention | | | | | |
|---|---|---|---|---|---|---|
| | 0 h | 1 h | 2 h | 4 h | 6 h | 8 h |
| D5 (comparative) | 100.0 | 25.1 | 19.6 | 16.4 | 15.5 | 15.1 |
| Working Sample D | 100.0 | 94.2 | 93.3 | 92.9 | 92.9 | 92.1 |
| Working Sample E | 100.0 | 88.3 | 87.3 | 86.9 | 86.0 | 85.6 |
| Working Sample F | 100.0 | 95.1 | 94.3 | 93.4 | 93.0 | 91.8 |
| Working Sample G | 100.0 | 93.2 | 92.2 | 91.3 | 91.1 | 90.7 |
| Working Sample H | 100.0 | 91.7 | 90.2 | 90.0 | 89.8 | 88.9 |

D5 control was statistically different than all silanes under the conditions tested. Observation: The higher the water ratio was, the higher the curl retention percentage was under the conditions tested.

Additional samples were prepared according to the method of Synthesis Example 1 above, except different ratios of alkyltriethoxysilane to aminopropyltriethoxysilane were used, and different alkyltriethoxysilanes were used (e.g., methyltriethoxysilane or isobutyltriethoxysilane, as shown by “methyl” or “isobutyl”, respectively, in Table 4).

TABLE 4
Materials

| Name | Product |
|---|---|
| D5 (comparative) | decamethylcyclopentasiloxane from DSC |
| Working Sample I | Water Ratio 3 |
| | (70:20 Methyl:Amino) |
| | Carbamate level 0.4% |
| Working Sample E | Water Ratio 3 |
| | (60:30 Methyl:Amino) |
| | Carbamate level 0.4% |
| Working Sample J | Water Ratio 3 |
| | (60:30 Isobutyl:Amino) |
| | Carbamate level 0.4% |
| Working Sample K | Water Ratio 3 |
| | (30:30:30 Methyl:Isobutyl:Amino) |
| | Carbamate level 0.4% |
| Working Sample L | Water Ratio 3 |
| | (50:40 Methyl:Amino) |
| | Carbamate level 0.4% |

TABLE 5
Results of Curl Retention Testing of Materials in Table 4

| | % curl retention | | | | | |
|---|---|---|---|---|---|---|
| | 0 h | 1 h | 2 h | 4 h | 6 h | 8 h |
| D5 | 100.0 | 28.4 | 22.4 | 19.3 | 17.6 | 17.0 |
| Working Sample I | 100.0 | 93.6 | 92.9 | 90.6 | 88.9 | 87.4 |
| Working Sample E | 100.0 | 86.1 | 85.0 | 83.7 | 83.1 | 81.8 |
| Working Sample J | 100.0 | 62.9 | 56.2 | 38.2 | 34.4 | 31.7 |
| Working Sample K | 100.0 | 88.5 | 87.7 | 86.4 | 85.8 | 85.2 |
| Working Sample L | 100.0 | 84.2 | 80.2 | 77.9 | 76.7 | 71.6 |

D5 control was statistically different than all the silanes under the conditions tested. All of the working examples in Table 5 showed better curl retention than the D5 control. The higher the methyl level was, the higher the curl retention percentage was under the conditions tested.

### Test Methods

¹³C NMR spectroscopy was performed as follows: The measurement was performed using a 600 MHz AVANCE NEO NMR spectrometer from BRUKER. 1.5 g of the sample was solubilized in 3.5 g CDCl₃ as solvent, with addition of Cr(acac)₃ to reduce the relaxation. The probe was a 10 mm broad band probe. As NMR technique the Inverse Gated Pulse sequence was applied. Using the 90° pulse, the relaxation delay was set to 15 s. The number of scans was selected to be 1300. The ¹³C NMR spectrum showed the carbamate peak at 162-164 ppm.

Near IR (NIR) evaluation was performed as follows: Transparent liquid samples were transferred into glass vials and then capped (8 mm outer diameter), and then their spectra were measured in transmission mode using an ABB MB3600 FT-NIR instrument. Sample temperature was kept at 30° C. using a heating block. Instrument configuration: 8 cm⁻¹ resolution, 4,000˜15,000 cm⁻¹ spectral range, 32 scans averaged per spectrum.

ATR-FTIR evaluations were performed as follows: A Thermo-Nicolet 6700 FTIR instrument equipped with a single-bounce diamond attenuated total reflectance (ATR) crystal was used to acquire sample spectra in the ATR mode. Samples were analyzed as received by placing a drop onto the ATR crystal followed by immediate spectral collection to minimize any potential atmospheric CO₂ uptake. Instrument configuration: 4 cm⁻¹ resolution, 400˜4,000 cm⁻¹ spectral range, 32 scans averaged per spectrum.

Cure speed was tested using a rheometer, Anton Parr MCR300, equipped with a Spindle CP50. Measurement details used are described below.
Oscillation measurement
Constant stain 100%
Constant frequency 1 Hz
5000 points measured
Spindle cone plate 50 mm
Gap of 0.049 mm
0.7 mL of material applied
Temperature 38° C.
Time 100 minutes

Complex viscosity was measured according to A Basic Introduction to Rheology (2016 Malvern Instruments Limited) WP160620BasicIntroRheology, as follows: a complex viscosity η*is a measure of the total resistance to flow as a function of angular frequency (ω) and is given by the quotient of the maximum stress amplitude and maximum strain rate amplitude.

$${η}^{*} = {G}^{*} / ω$$

As with G*this can be broken down into its component parts, which include the dynamic viscosity (η′) and the storage viscosity (η″), which represent the real and imaginary parts of η*respectively.

Long Lasting Frizz Index was evaluated as follows:
Hair type: “frizzy” type A tress weight ˜4 g
Product quantity: ˜0.03 g of product applied per g of hair
Treatment: Silane blend diluted at 25% in D5
Conditions: Relative Humidity (RH)=80%, T°=25° C.
Test protocol:
Prewash tresses with SLS solution (9%)
Treat tresses with 0.05, 0.1 or 0.15 g per g of tresses
Apply dilution on wet hair and massage the tresses
Make a brushing with a brush using a hair drier
Apply the iron plate 12 times
Take pictures at the several times (T0-30-60-90 min)

Aspect Ratio was calculated from the values obtained as described above for long lasting frizz index and represents the length of the hair tress over the width.

Curl Retention Percentage was evaluated as follows:
Hair type: Dark Brown 2 g/20 cm gross ½″ wide
Product quantity: 100 μL of product applied per tress
Treatment: Samples A, B, and C were diluted at 25% in D5
Conditions: Relative Humidity (RH)=80%, T°=25° C.
Test protocol:
   Wet hair tresses for 30 s with tap water at 37° C.
   Lather the hair tresses for 30 sec. Apply 1 g of 30% SLS solution per hair tress and stroke the tress downward.
   Leave the surfactant to act for 30 sec.
   Rinse the hair tresses for 1 minute with tap water at 37° C.
   Remove the excess of water by running three times the tresses between the two fingers.
   Allow the tresses to dry overnight on a paper towel (RT).
   Apply 100 μL of treatment per gram of hair. Leave them to hang for 5 minutes.
   Curl each tress following the below procedure:
   Curling procedure:
a.\Detangle the tress completely
b.\Roll the tress on curler.
c.\Leave the hair swatches to dry overnight in an oven at 40° C.
d.\Carefully remove the curler from hair by twisting it slightly, 10 minutes before the start of the test.
e.\Test them for curl retention by measuring the evolution of hair tress length at different time intervals when submitted to specific temperature and humidity conditions.

### INDUSTRIAL APPLICABILITY

The carbamate anion/protonated amine-functional silane hydrolyzate described herein can be used in a hair care composition. The carbamate anion/protonated amine-functional silane hydrolyzate, when used in the hair care composition, may provide styling, film forming and/or hair manageability benefits including (but not limited to) frizz control and curl retention.

U.S. Pat. No. 9,962,327 discloses a cosmetic or dermatological composition of sol/gel type for making up and/or caring for keratin materials. This composition is obtained by sol/gel reaction by mixing at least one alkoxysilane and at least one specific amount of water, determined according to the number of moles of alkoxy groups in the mixture. The alkoxysilane may have an amino group.

However, the presence of an amino-functional alkoxysilane compound and/or an excessive amount of amino groups in a hydrolyzate can impart undesirable odor to cosmetic or dermatological compositions, such as those used for hair care applications. In addition, amino groups and amino-functional alkoxysilane compounds may act as skin irritants and/or sensitizers. Therefore, a problem to be addressed by this invention is to meet the need in the silicones industry for silane hydrolyzates with reduced amino content suitable for use in cosmetic or other personal care compositions that may contact skin, such as hair care compositions that may also contact skin, e.g., the scalp.

The silane hydrolyzate prepared in step (1) of the process described herein and in claim 1 below may contain relatively high amounts of residual amino-functional alkoxysilane compound and amino-functional groups on the species of unit formula (I-2), shown above. For example, Synthesis Example 1 and Working Example B above show that after step (1), the silane hydrolyzate may contain 2.5% APTES and 2.2% amino-propyl groups on the species of unit formula (I-1). Step (2) of the process drastically reduces the amino content of the carbamate anion/protonated amine-functional silane hydrolyzate. For example, after step (2) the carbamate anion/protonated amine-functional silane hydrolyzate contained only 0.5% APTES and 0.44% amino-propyl groups on the species of unit formula (I-2). Without wishing to be bound by theory, it is thought that the carbamate anion/protonated amine-functional silane hydrolyzate will have less potential to create undesirable odor, skin irritation and/or skin sensitization than silane hydrolyzates produced according to U.S. Pat. No. 9,962,327.

### Definitions and Usage of Terms

All amounts, ratios, and percentages herein are by weight, unless otherwise indicated. The articles ‘a’, ‘an’, and ‘the’ each refer to one or more, unless otherwise indicated by the context of specification. The singular includes the plural unless otherwise indicated. The SUMMARY and ABSTRACT are hereby incorporated by reference. The transitional phrases “comprising”, “consisting essentially of”, and “consisting of” are used as described in the Manual of Patent Examining Procedure Ninth Edition, Revision 08.2017, Last Revised January 2018 at section § 2111.03 I., II., and III. Any feature, embodiment, or aspect of the invention may be used in combination with any other feature, embodiment, or aspect recited herein. The abbreviations used herein have the definitions in Table 3.

TABLE 3
Abbreviations

| Abbreviation | Definitions |
|---|---|
| acac | acetyl acetonate |
| ° C. | degrees Celsius |
| cm | centimeter |
| FTIR | Fourier transform infra-red |
| g | gram |
| h | hour |
| Hz | Hertz |
| Me | methyl |
| min | minute |
| mL | milliliter |
| mm | millimeter |
| NMR | nuclear magnetic resonance |
| Ph | phenyl |
| ppm | parts per million by weight |
| RPM | revolutions per minute |
| RT | room temperature of 25 ± 5° C. |
| μm | micrometer |
| Vi | vinyl |

### Embodiments of the Invention

In a first embodiment, a CAPA-functional silane hydrolyzate comprises a species of unit formula: (R³Z₂SiO_{1/2})ₑ(R⁵Z₂SiO_{1/2})_{f}(R³ZSiO_{2/2})_{g}(R⁵ZSiO_{2/2})ₕ(R³SiO_{3/2})ᵢ(R⁵SiO_{3/2})ⱼ, where
each R³ is independently selected from the group consisting of a monovalent hydrocarbon group and a monovalent halogenated hydrocarbon group;
each Z is an alkoxy group of formula OR², where each R² is an independently selected alkyl group;
each R⁵ is independently selected from the group consisting of amino-alkyl, carbamate-alkyl, and protonated amine alkyl, with the proviso that at least one instance of R⁵ being a carbamate-alkyl and at least one instance of R⁵ being a protonated amine alkyl are present in the CAPA-functional silane hydrolyzate;
subscripts e, f, g, h, i, and j represent average numbers of each unit per molecule and have values such that
   e≥0,
   f≥0,
   g≥0,
   h≥0,
   i≥0,
   j≥0, and
   a quantity (e+f+g+h+i+j)≥2,
   a quantity (e+g+i)>0, and
   a quantity (f+h+j)>0;
with the proviso that the CAPA-functional silane hydrolyzate is substantially free of siloxane units of formula (SiO₄/2).

In a second embodiment, in the CAPA-functional silane hydrolyzate of the first embodiment, for R⁵ the amino-alkyl group is aminopropyl, the carbamate-alkyl group is

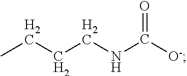

and the protonated amine group is

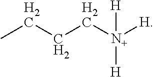

In a third embodiment, a process for preparing a CAPA-functional silane hydrolyzate comprises:

(1) combining, at a temperature up to 85° C., starting materials (A), (B), (C), and (D), wherein
starting material (A) is an amino-functional alkoxysilane compound of formula R¹Si(OR²)₃, where R¹ is an amino-alkyl group, and each R² is an independently selected alkyl group;
starting material (B) is an organoalkoxysilane compound of formula R³Si(OR²)₃, where each R³ is independently selected from the group consisting of a monovalent hydrocarbon group and a monovalent halogenated hydrocarbon group, and each R² is an independently selected alkyl group;
where (A) the amino-functional alkoxysilane compound and (B) the organoalkoxysilane compound are used in a weight ratio (B)/(A) of 1 to 3.5;
starting material (C) is a base catalyst; and
starting material (D) is water, where (D) the water is used in an amount corresponding to a number of moles determined according to the following equation

$$X = ( {n}_{alkoxysilane} , {xnb}_{alkoxygroups} ) / {n}_{H 2 O} ,$$

n_{H2O}=number of moles of water,
n_{Alkoxysilanes}=number of moles of alkoxysilane compounds (A) and (B);
nb_{alkoxygroups}=weighted mean of the number of alkoxysilane groups per alkoxysilane compound, and
X≥2.5;
thereby forming a silane hydrolyzate comprising unreacted (A) amino-functional alkoxysilane compound and/or unreacted (B) organoalkoxysilane compound, a siloxane oligomer, and an alcohol; and
   (2) combining, at a temperature up to 85° C., the silane hydrolyzate and (E) carbon dioxide, thereby neutralizing (C) the base catalyst and forming a reaction product comprising the CAPA-functional silane hydrolyzate.

In a fourth embodiment, in the process of the third embodiment, in (A) the amino-functional alkoxysilane compound, R¹ has formula —(C_{c}H_{2c})NH₂, where subscript c is 1 to 20; and R² has formula —C_{d}H_{(2d+1)}, where subscript d is 1 to 10.

In a fifth embodiment, in the process of the third or fourth embodiment, (A) the amino-functional alkoxysilane compound is selected from the group consisting of aminopropyltrimethoxysilane, amino-propyltriethoxysilane, and a combination of two or more thereof.

In a sixth embodiment, in the process of any one of the third to fifth embodiments, in (B) the organoalkoxysilane compound, R³ is an alkyl group of 1 to 20 carbon atoms; and R² has formula —C_{d}H_{(2d+1)}, where subscript d is 1 to 10.

In a seventh embodiment, in the process of the sixth embodiment, (B) the organoalkoxysilane compound is selected from the group consisting of methyltrimethoxysilane, ethyltrimethoxysilane, methyltriethoxysilane, ethyltriethoxysilane, propyltrimethoxysilane, propyltriethoxysilane, isobutyltrimethoxysilane, isobutyltriethoxysilane, and a combination of two or more thereof.

In an eighth embodiment, in the process of any one of the third to seventh embodiments, (C) the base catalyst is selected from the group consisting of a metal hydroxide and an alkyl ammonium hydroxide.

In a ninth embodiment, in the process of the eighth embodiment, the metal hydroxide is selected from the group consisting of KOH, NaOH, and a combination thereof.

In a tenth embodiment, in the process of the eighth embodiment, the alkyl ammonium hydroxide is selected from the group consisting of trimethylammonium hydroxide, cetyltrimethylammonium hydroxide, and a combination thereof.

In an eleventh embodiment, in the process of any one of the third to tenth embodiments, X is 2.5 to 500.

In a twelfth embodiment, in the process of the eleventh embodiment, X is 2.5 to 2.5 to 50.

In a thirteenth embodiment, in the process of the eleventh embodiment, X is 2.5 to 4.11.

In a fourteenth embodiment, in the process of the eleventh embodiment, X is 3 to 4.11.

In a fifteenth embodiment, in the process of the eleventh embodiment, X is 3.5 to 4.11.

In a sixteenth embodiment, in the process of any one of the third to fifteenth embodiments, the process further comprises dissolving (C) the base catalyst in (D) the water before combining (C) the base and (D) the water with (A) the amino-functional alkoxysilane compound and (B) the organoalkoxysilane compound.

In a seventeenth embodiment, in the process of any one of the third to sixteenth embodiments, where the temperature in step 1) is 50° C. to 85° C.

In an eighteenth embodiment, in the process of any one of the third to seventeenth embodiments, the temperature in step 1) is at least 70° C.

In a nineteenth embodiment, in the process of any one of the third to eighteenth embodiments, the temperature in step 2) is 50° C. to 85° C.

In a twentieth embodiment, in the process of any one of the third to nineteenth embodiments, the temperature in step 2) is at least 70° C.

In a twenty-first embodiment, a carbamate anion/protonated amine-functional silane hydrolyzate is prepared by the process of any one of the third to twentieth embodiments.

In a twenty-second embodiment, a hair care composition comprises: (I) the carbamate anion/protonated amine-functional silane hydrolyzate of the twenty-first embodiment, and (II) a cyclic polydiorganosiloxane.

In a twenty-third embodiment, the carbamate anion/protonated amine-functional silane hydrolyzate of the twenty-first embodiment is used in a hair care composition for frizz control and/or curl retention.

In a twenty-fourth embodiment, the carbamate anion/protonated amine-functional silane hydrolyzate of the twenty-first embodiment is used as a film forming agent.

1. A process for preparing a carbamate anion/protonated amine-functional silane hydrolyzate, wherein the process comprises:
(1) combining, at a temperature up to 85° C., starting materials (A), (B), (C), and (D), wherein
starting material (A) is an amino-functional alkoxysilane compound of formula R¹R³₍₃₋ₐ₎Si(OR²)ₐ, where each R¹ is an independently selected amino-alkyl group, each R² is an independently selected alkyl group, each R³ is independently selected from the group consisting of a monovalent hydrocarbon group and a monovalent halogenated hydrocarbon group, and subscript a is an integer from 1 to 3;
starting material (B) is an organoalkoxysilane compound of formula R³_{(4-b)}Si(OR²)_{b}, where each R³ is independently selected from the group consisting of a monovalent hydrocarbon group and a monovalent halogenated hydrocarbon group, each R² is an independently selected alkyl group, and subscript b is an integer from 1 to 3;
where (A) the amino-functional alkoxysilane compound and (B) the organoalkoxysilane compound are used in a weight ratio (B)/(A) of 1 to 3.5;
starting material (C) is a base catalyst; and
starting material (D) is water, where (D) the water is used in an amount corresponding to a number of moles determined according to the following equation
$$X = ( {n}_{alkoxysilane} , {xnb}_{alkoxygroups} ) / {n}_{H 2 O} ,$$
n_{H2O}=number of moles of water,
n_{Alkoxysilanes}=number of moles of alkoxysilane compounds (A) and (B);
nb_{alkoxygroups}=weighted mean of the number of alkoxysilane groups per alkoxysilane compound, and
X≥2.5;
thereby forming a silane hydrolyzate comprising unreacted (A) amino-functional alkoxysilane compound and/or unreacted (B) organoalkoxysilane compound, a siloxane oligomer, and an alcohol; and
(2) combining, at a temperature up to 85° C., the silane hydrolyzate and (E) carbon dioxide, thereby neutralizing (C) the base catalyst and forming a reaction product comprising the carbamate anion/protonated amine-functional silane hydrolyzate.

2. The process of claim 1, where in (A) the amino-functional alkoxysilane compound, R¹ has formula —(C_{c}H_{2c})NH₂, where subscript c is 1 to 20; R² has formula —C_{d}H_{(2d+1)}, where subscript d is 1 to 10; and subscript a is 2 or 3.

3. The process of claim 2, where (A) the amino-functional alkoxysilane compound is selected from the group consisting of aminopropyltrimethoxysilane, amino-propyltriethoxysilane, amino-propyl, methyl, dimethoxysilane, amino-propyl, methyl, diethoxysilane, and a combination of two or more thereof.

4. The process of claim 1, where in (B) the organoalkoxysilane compound, R³ is an alkyl group of 1 to 20 carbon atoms; R² has formula —C_{d}H_{(2d+1)}, where subscript d is 1 to 10; and subscript b is 2 or 3.

5. The process of claim 4, where (B) the organoalkoxysilane compound is selected from the group consisting of methyltrimethoxysilane, ethyltrimethoxysilane, methyltriethoxysilane, ethyltriethoxysilane, propyltrimethoxysilane, propyltriethoxysilane, butyltrimethoxysilane, butyltriethoxysilane, and a combination of two or more thereof.

6. The process of claim 1, where (C) the base catalyst is selected from the group consisting of a metal hydroxide and an alkyl ammonium hydroxide.

7. The process of claim 6, where the metal hydroxide is selected from the group consisting of KOH, NaOH, and a combination thereof; and the alkyl ammonium hydroxide is selected from the group consisting of trimethylammonium hydroxide, cetyltrimethylammonium hydroxide, and a combination thereof.

8. The process of claim 1, where X is 2.5 to 500, alternatively 2.5 to 2.5 to 50, alternatively 2.5 to 4.11, alternatively 3 to 4.11, and alternatively 3.5 to 4.11.

9. The process of any one of claim 1, where the method further comprises dissolving the base catalyst in water before combining the base catalyst with the amino-functional alkoxysilane compound and the organoalkoxysilane compound.

10. The process of claim 1, where the temperature in step 1) is 50° C. to 85° C.; or the temperature in step 2) is 50° C. to 85° C.; or both.

11. The process of claim 1, wherein the carbamate anion/protonated amine-functional silane hydrolyzate comprises unit formulae:
(R³Z₂SiO_{1/2})ₑ(R⁵Z₂SiO_{1/2})_{f}(R³ZSiO_{2/2})_{g}(R⁵ZSiO_{2/2})ₕ(R³SiO_{3/2})ᵢ(R⁵SiO_{3/2})ⱼ(R³R⁵ZSiO_{1/2})ₖ(R³R⁵SiO_{2/2})ₘ(R³₂ZSiO_{1/2})ₙ(R³₂SiO_{2/2})ₒ(R³₃SiO_{1/2})ₚ(R³₂R⁵SiO_{1/2})_{q},
R³ is as defined above;
each Z is an alkoxy group of formula OR², where R² is as defined above;
each R⁵ is independently selected from the group consisting of amino-alkyl, carbamate-alkyl, and protonated amine alkyl, with the proviso that at least one instance of R⁵ being a carbamate-alkyl and at least one instance of R⁵ being a protonated amine alkyl are present in the carbamate anion/protonated amine-functional silane hydrolyzate;
subscripts e, f, g, h, i, j, k, m, n, and o represent average numbers of each unit per molecule and have values such that
e≥0,
f≥0,
g≥0,
h≥0,
i≥0,
j≥0,
k≥0,
m≥0,
n≥0,
o≥0,
p≥0,
q≥0, and
a quantity (e+f+g+h+i+j+k+m+n+o+p+q+)≥2;
with the proviso that the carbamate anion/protonated amine-functional silane hydrolyzate is substantially free of siloxane unites of formula (SiO_{4/2}).

12. The process of claim 11, where R⁵ the amino-alkyl group is aminopropyl, the carbamate-alkyl group is 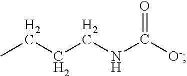
and the protonated amine group is

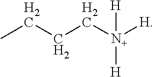

13. A carbamate anion/protonated amine-functional silane hydrolyzate prepared by the process of claim 1.

14. A hair care composition comprising:
(I) the carbamate anion/protonated amine-functional silane hydrolyzate of claim 11, and
(II) a cyclic polydiorganosiloxane.

15. (canceled)

16. The method of claim 1, where subscript b=2 or 3.

17. The method of claim 1, where the A) the amino-functional alkoxysilane compound is an amino-functional trialkoxysilane, and when (B) the organoalkoxysilane compound is an organotrialkoxysilane.

18. The method of claim 17, where the carbamate anion/protonated amine-functional silane hydrolyzate comprises species each having unit formula (I-2):
(R³Z₂SiO_{1/2})ₑ(R⁵Z₂SiO_{1/2})_{f}(R³ZSiO_{2/2})_{g}(R⁵ZSiO_{2/2})ₕ(R³SiO_{3/2})ᵢ(R⁵SiO_{3/2})ⱼ, where
each R³ is independently selected from the group consisting of a monovalent hydrocarbon group and a monovalent halogenated hydrocarbon group;
each Z is an alkoxy group of formula OR², where each R² is an independently selected alkyl group;
each R⁵ is independently selected from the group consisting of amino-alkyl, carbamate-alkyl, and protonated amine alkyl, with the proviso that at least one instance of R⁵ being a carbamate-alkyl and at least one instance of R⁵ being a protonated amine alkyl are present in the carbamate anion/protonated amine-functional silane hydrolyzate;
subscripts e, f, g, h, i, and j represent average numbers of each unit per molecule and have values such that e≥0, f≥0, g≥0, h≥0, i≥0, j≥0, a quantity (e+f+g+h+i+j)≥2, a quantity (e+g+i)>0, and a quantity (f+h+j)>0.

19. The method of claim 18, where the carbamate anion/protonated amine-functional silane hydrolyzate comprises a combination of different species including:
i) a dimer where in the unit formula (I-2) above subscripts g=h=i=j=0 and the dimer has unit formula (I-3): (R³Z₂SiO_{1/2})ₑ(R⁵Z₂SiO_{1/2})_{f}, where subscript e is 0 or 1, subscript f is 1 or 2, and a quantity (e+f)=2; and
ii) a linear trimer where in the unit formula (I-2) above subscripts i=j=0, and the linear trimer has unit formula (I-4):
(R³Z₂SiO_{1/2})ₑ(R⁵Z₂SiO_{1/2})_{f}(R³ZSiO_{2/2})_{g}(R⁵ZSiO_{2/2})ₕ, where subscript e is 0, 1, or 2; subscript f is 0, 1, or 2; a quantity (e+f)=2, a quantity (g+h)=1, and a quantity (f+h)>0.
